(19) United States
(12) Patent Application Publication
(10) Pub. No.: US 2025/0207196 A1
(43) Pub. Date: Jun. 26, 2025

(54) USE OF THE RELATIVE GENE EXPRESSION LEVEL VALUE OF GENE HMGA2 IN THE PROGNOSIS OR DIAGNOSIS OF A FATTY LIVER DISEASE OF A SUBJECT

(71) Applicant: Lipocyte BioMed GmbH, Bremen (DE)

(72) Inventors: Wolfgang HIERNEIS, Garmisch-Partenkirchen (DE); Uwe JENSEN, Bremen (DE); Markus KLEMKE, Rosengarten (DE); Helge Wilhelm THIES, Bremen (DE)

(73) Assignee: Lipocyte BioMed GmbH, Bremen (DE)

(21) Appl. No.: 18/847,604

(22) PCT Filed: Mar. 15, 2023
(86) PCT No.: PCT/EP2023/056573

§ 371 Date: Sep. 16, 2024

(30) Foreign Application Priority Data
Mar. 16, 2022 (DE) 10 2022 106 198.1

(51) Int. Cl. C12Q 1/6883

(52) U.S. Cl. CPC C12Q 1/6883; C12Q 2600/118; C12Q 2600/158

(57) ABSTRACT

The use of the relative value of the gene expression level of the gene HMGA2 and/or of a gene, the expression of which statistically correlates with that of the gene HMGA2, in the prognosis and/or diagnosis of a fatty liver disease of a subject.

The present invention relates to the use of the relative value of the gene expression level of the gene HMGA2 and/or of a gene, the expression of which statistically correlates with that of the gene HMGA2, in the prognosis and/or diagnosis of a fatty liver disease of a subject. The invention relates in particular to a corresponding use for distinguishing between a nonalcoholic fatty liver disease (NAFLD) and a nonalcoholic steatohepatitis (NASH) and to a corresponding use for prognosing the deterioration of the state of the liver.

The invention further relates to a method for prognosing and/or diagnosing a liver disease.

Nonalcoholic fatty liver disease (NAFLD) is the most common chronic liver disease in Germany with increasing prevalence. NAFLD includes in particular steatosis hepatis (fatty liver) and is a precursor of nonalcoholic steatohepatitis (NASH; i.e., fatty liver with inflammation and liver cell necrosis) and liver cirrhosis. NAFLD occurs in all age groups, and is found in 14% to 30% of the general population [Browning J, Szczepaniak L, Dobbins R, Nuremberg P, Horton J D, Cohen J C, Grundy S M, Hobbs H H. Prevalence of hepatic steatosisin an urban population in the United States: impact of ethnicity. Hepatology 2004; 40:1387-95; Nomura H, Kashiwagi S, Hayashi J, Kajiyama W, Tani S, Goto M. Prevalence of fatty liver in a general population of Okinawa, Japan. Jpn J Med 1988; 27:142-9.]. Prevalence increases yet further for patients of relatively high body weight. Steatosis hepatis can be diagnosed in about 66% of patients with a BMI ≥30 kg/m² and in over 90% of patients with a BMI of >39 kg/m² [Angulo P. Nonalcoholic fatty liver disease. N Engl J Med. 2002; 346:1221-31.]. Globally, the prevalence of NAFLD in countries with a Western lifestyle, such as Europe, USA, Latin America, China and Japan, is 20-30%. The prevalence of NASH in these countries is between 3-16%.

The pathogenesis of NAFLD is far from completely understood, but a “two hit model” hypothesis which leads to the development of NAFLD is taken as a basis. The accumulation of fat in the liver is closely linked to insulin resistance. The latter leads to activation of lipolysis in peripheral adipose tissues, thereby resulting in an increased influx of free fatty acids (FFA) into the liver. In addition, insulin resistance causes increased “de novo” synthesis of triglycerides and downregulation of β-oxidation within the liver, which then leads to intrahepatic accumulation of triglycerides (“first hit”) [Browning J D, Horton J D. Molecular mediators of hepatic steatosis and liver injury. J Clin Invest 2004; 114: 147-52.]. The “second hit” comprises mechanisms which lead to the development of inflammation and fibrosis [Day C P, James O F. Steatohepatitis: a tale of two “hits”? Gastroenterology 1998; 114: 842-5.]. Different factors such as oxidative stress, mitochondrial abnormalities and hormonal disorders are held responsible for this [Li Z, Yang S, Lin H, Huang J, Watkins P A, Moser A B, Desimone C, Song X Y, Diehl A M. Probiotics and antibodies to TNF inhibit inflammatory activity and improve nonalcoholic fatty liver disease. Hepatology 2003; 37: 343-50.; Haque M, Sanyal A J. The metabolic abnormalities associated with non-alcoholic fatty liver disease. Best Pract Res Clin Gastroenterol 2002; 16:709-31.].

Disease course and prognosis differ between NAFLD and NASH patients. Whereas NAFLD commonly has a mild disease course, NASH exhibits hepatic necroinflammation and increasing fibrosis which can progress as far as liver cirrhosis. In 5-20% of patients with NAFLD, the diseases progresses to NASH and in 10-20% of NASH patients, the disease progresses through fibrosis development toward liver cirrhosis (<5%) [Vernon G, Baranova A, Younossi Z M. Systematic review: the epidemiology and natural history of nonalcoholic fatty liver disease and non-alcoholic steatohepatitis in adults. Aliment Pharmacol Ther. 2011; 34: 274-85]. The incidence of hepatocellular carcinoma (HCC) in patients with NASH and cirrhosis is 2.6-11.3% [Baffy G, Brunt E M, Caldwell S H. Hepatocellular carcinoma in non-alcoholic fatty liver disease: an emerging menace. J Hepatol. 2012; 56:1384-91], but NAFLD patients without cirrhosis may also be affected by HCC. Owing to the increasing prevalence of NASH-liver cirrhosis in the USA, but also in Germany, this indication is, according to experts, the main reason for liver transplantation in the 2020s. Experts recommend that attention already be paid to NAFLD in its early disease stages in order to prevent NASH-associated sequelae.

Since the development of NAFLD is often associated with insulin resistance, it commonly occurs in metabolic disorders such as overweight, diabetes mellitus and hyperlipidemia. Most patients with NAFLD are clinically asymptomatic. Abnormalities determined by laboratory tests are often the only indications of NAFLD, the most common changes concerning an increase in gamma-GT in the case of purely fatty liver and an increase in transaminases (AST, ALT) in the case of NASH. Diagnosing NAFLD requires clear evidence of steatosis hepatis, which is supplied either by imaging methods (e.g., sonography, CT or MRI) or by liver biopsy. Abdominal sonography is a relatively inexpensive examination, and it is therefore commonly used as a screening method. However, the sensitivity is too low to be able to also detect minimal changes (<30%) in obese patients [Mottin C C, Moretto M, Padoin A V, Swarowsky A M, Toneto M G, Glock L, Repetto G. The role of ultrasound in the diagnosis of hepatic steatosis in morbidly obese patients. Obes Surg 2004; 14:635-7.]. Serologically, progressive fibrosis can be determined on the basis of the FIB4 score, which is calculated from age, thrombocyte count and transaminases (ALT, AST). The FIB4 score has a good negative predictive value for ruling out advanced fibrosis. However, the positive predictive value of the FIB4 score has not yet been clearly demonstrated scientifically. The gold standard for diagnosing NAFLD continues to be liver biopsy. Furthermore, biopsy is still the only examination method capable of distinguishing between simple steatosis and NASH and quantifying the extent of existing fibrosis [Saadeh S, Younossi Z M, Remer E M, Gramlich T, Ong J P, Hurley M, Mullen K D, Cooper J N, Sheridan M J. The utility of radiological imaging in nonalcoholic fatty liver disease. Gastroenterology 2002; 123: 745-50.]. However, its routine use is highly contentious because NAFLD usually has a benign course, no established therapies for NAFLD are available, and in particular, however, biopsy itself is a diagnostic method which involves considerable risks [Dienstag J L. The role of liver biopsy in chronic hepatitis C. Hepatology 2002; 36 (Suppl 1): S152-60.]. This is because a liver biopsy is not only associated with a substantial morbidity rate (postoperative pain in 10-30%), but also associated with the risk of deterioration of liver fibrosis and even with a certain mortality rate (approx. 0.1-0.01%) [Stauber R. Nichtinvasive Diagnose der Leberfibrose bei chronischen Hepatopathien [Noninvasise diagnosis of liver fibrosis in chronic hepatopathies]. J Gastroenterol Hepatol Erkr 2019; 7:12-17]. Furthermore, a fundamental problem is that the histopathological structures of NAFLD/NASH may have a nonuniform distribution in the liver, meaning that histological diagnosis on the basis of liver biopsy may be associated with a substantial sampling error [Ratziu V, et al. Sampling variability of liver biopsy in nonalcoholic fatty liver disease. Gastroenterology 2005; 128: 1898-1906].

Therapy for NAFLD is currently mainly focused on the components of metabolic syndrome, in particular insulin resistance, since steatosis hepatis alone is unlikely to justify treatment. Attempts at therapy to date have concentrated on various aspects of the disease. This included focusing on identifying and treating underlying metabolic disorders, such as diabetes mellitus and hyperlipidemia. With these patients, attempts were made to improve insulin sensitivity by weight reduction, by increased physical activity or by various drug therapies. At the same time, liver-protective substances such as antioxidants were used to protect the liver from the “second hit”. The majority of patients with NAFLD are obese with increased visceral fat mass, which is associated with increased insulin resistance. For such patients, weight reduction is therefore the logical “first line” therapy. However, weight should not fall by more than 0.5-1 kg per week, since excessively rapid weight loss (e.g., after a bariatric procedure) can lead to distinct deterioration of existing steatohepatitis [Andersen T, Gluud C, Franzmann M B, Christoffersen P. Hepatic effects of dietary weight loss in morbidly obese subjects. J Hepatol 1991; 12: 224-9.; Luyckx F H, Scheen A J, Desaive C, Dewe W, Gielen J E, Lefebvre P J. Effects of gastroplasty on body weight and related biological abnormalities in morbid obesity. Diabetes Metab 1998; 24: 355-61.]

Proceeding from the prior art, it is an object of the present invention to specify a method for reliable diagnosis and/or prognosis of liver diseases. In particular, the aim is to determine reliable diagnosis and prognosis values without the need for a (risky) liver biopsy. Furthermore, it is preferred that diagnosis involves distinguishing particular forms of liver disease from one another, and it is also preferred that prognosis involves allowing statements about potential progression of pre-existing liver diseases.

This object is achieved by the use of the relative value of the gene expression level of the gene HMGA2 and/or of a gene, the expression of which statistically correlates with that of the gene HMGA2, in the prognosis and/or diagnosis of a fatty liver disease of a subject.

It has been found that, surprisingly, the gene expression of HMGA2 in adipose tissue is associated with the progression of liver fibrosis and plays an important role in distinguishing between the severity levels of liver damage and also in making a classification between NAFLD and NASH (see also below). This connection between HMGA2 expression and liver diseases is completely new and has not been scientifically described.

“HMGA2” in the context of this text is the High Mobility Group AT-Hook Protein 2 (HMGA2) or the gene thereof or the associated mRNA and/or parts of this protein or gene (or the mRNA thereof), preferably at least one amino acid chain ≥7 amino acids, more preferably ≥15 amino acids and particularly preferably ≥20 amino acids or a nucleic acid chain of ≥20 nucleic acids, more preferably ≥40 nucleic acids and particularly preferably ≥55 nucleic acids, per strand as applicable. HMGA2 is a transcription factor which influences the regulation of gene expression and belongs to the group of the High Mobility Group A proteins (HMGA proteins). The HMGA proteins are chromatin-associated, acid-soluble non-histone proteins which bind to sequence-independent, specific motifs of DNA. As architectural transcription factors, they increase or inhibit the binding capacity of other transcription factors through structural changes in chromatin organization. The human HMGA2 gene is located in chromosome region 12q14˜15 and consists of five exons extending over a region ≥160 kb in length. It encodes a protein of 109 amino acids in length with a molecular mass of 12 kDa. The HMGA2 protein is characterized by three highly conserved DNA-binding domains, the so-called AT hooks, and an acidic, negatively charged C-terminal domain.

In the context of this text, a gene, the expression of which “statistically correlates” with that of another (stated) gene, is one in which there is a mathematical relationship between the gene expression of the gene in question and that of the stated gene. Preferably, there is linear correlation of the expression of these two genes in question.

The value for the relative gene expression level in the context of the present invention can be determined in any manner known to a person skilled in the art. Preference is given to determination of the gene expression level at the mRNA level or at the protein level. Preference is given to the mRNA level.

In the present invention, relative gene expression levels are determined. Where mention is only made of “gene expression levels” hereinafter, this always means relative gene expression levels, unless otherwise stated. The relative gene expression levels are preferably determined by determining the gene expression level of the gene to be investigated in relation to the expression level of a housekeeping gene, preferably selected from the group consisting of HPRT, 18S rRNA, GAPDH, GUSB, PBGD, B2M, ABL, RPLP0, with very particular preference being given to HPRT.

A preferred method for determining the relative gene expression level is described in Schmittgen, Thomas D and Livak, Kenneth J, 2008, Analyzing real-time PCR data by the comparative C_{T} method (Nature Protocols 3: 1001-1008).

In the context of this text, the term “prognosis” means a prediction of an increased probability of the development or occurrence of a clinical state or disease.

In the context of this text, the term “diagnosis” of a disease means that a disease already showing clinical symptoms is identified and/or confirmed.

Nonalcoholic fatty liver disease is also abbreviated as NAFLD in the text hereinafter. Nonalcoholic steatohepatitis is also abbreviated as NASH in the text hereinafter.

“Subject” in the context of the present application are humans and animals, the preferred meaning being humans.

Preference according to the invention is given to a use according to the invention, wherein the relative value of the gene expression level of the gene HMGA2 and/or of a gene, the expression of which statistically correlates with that of the gene HMGA2, is related to the relative value of the gene expression level of the gene ADIPOQ and/or of a gene, the expression of which correlates with that of the gene ADIPOQ.

“Adiponectin” (ADIPOQ) in the context of this text is accordingly the adiponectin protein or the gene thereof or the associated mRNA and/or parts of this protein or gene (or the mRNA thereof), preferably at least one amino acid chain ≥7 amino acids, more preferably ≥15 amino acids and particularly preferably ≥20 amino acids or a nucleic acid chain of ≥20 nucleic acids, more preferably ≥40 nucleic acids and particularly preferably ≥55 nucleic acids, per strand as applicable. Adiponectin is an important adipokine involved in the control of fat metabolism and insulin sensitivity and has a direct antidiabetic, antiatherogenic and antiinflammatory influence. It stimulates AMPK phosphorylation and activation in the liver and skeletal muscle, thereby increasing the utilization of glucose and the burning of fatty acids. The human adiponectin gene is located in chromosome region 3q27 and consists of three exons extending over a region ≥17 kb in length. It encodes, inter alia, a complete adiponectin protein of 244 amino acids in length with a molecular mass of 30 kDa. The adiponectin protein is characterized by a carboxy-terminal globular domain and a collagen domain at the amino-terminal end. Basically, adiponectin occurs in plasma as a complete protein (244 amino acids) and as a proteolytic cleavage-product fragment, also called globular adiponectin. The isoforms of adiponectin arise owing to different linkages between the globular and collagen domains. Three main complexes in particular circulate in plasma, a low-molecular-weight trimer (LMW), a medium-molecular-weight hexamer (MMW) and a high-molecular-weight complex (HMW).

“Relating” in the context of this text means that the combination of “related” values yields an additional gain of knowledge. “Relating” in the context of the present invention can in particular mean a comparison with already created databases or performance of a statistical method. A preferred statistical method is described below.

It has been found that, in the use according to the invention, the inclusion of the relative value of the gene expression level of the gene ADIPOQ leads to an improved prognostic or diagnostic result.

Preference is given to a use according to the invention, wherein additionally the relative value (i) of the gene expression level of the gene HMGA2 or (ii) of the gene HMGA2 and of the gene ADIPOQ is related to the relative value of one gene expression level or preferably both gene expression levels of the genes selected from the group consisting of PPAR-gamma and IL-6.

“PPAR-gamma” in the context of this text is the peroxisome proliferator-activated receptor gamma (PPARgamma), preferably PPAR-gamma isoform 2, or the gene thereof or the associated mRNA and/or parts of this protein or gene (or the mRNA thereof), preferably at least one amino acid chain ≥7 amino acids, more preferably ≥15 amino acids and particularly preferably ≥20 amino acids or a nucleic acid chain of ≥20 nucleic acids, more preferably ≥40 nucleic acids and particularly preferably ≥55 nucleic acids, per strand as applicable. PPAR-gamma is a ligand-binding nuclear transcription factor of the PPAR subfamily which belongs to the group of nuclear hormone receptors. PPAR-gamma activates the transcription of various genes via heterodimerization with the retinoid X receptor a (RXRa). The human PPAR-gamma gene is located in chromosome band 3p25 and consists of 11 exons. The human PPAR-gamma gene encodes 3 isoforms, which are a protein of 477 amino acids in length, a protein of 505 amino acids in length and a protein of 186 amino acids in length.

“IL-6” in the context of this text is interleukin-6 or the gene thereof or the associated mRNA and/or parts of this protein or gene (or the mRNA thereof), preferably at least one amino acid chain ≥7 amino acids, more preferably ≥15 amino acids and particularly preferably ≥20 amino acids or a nucleic acid chain of ≥20 nucleic acids, more preferably ≥40 nucleic acids and particularly preferably ≥55 nucleic acids, per strand as applicable. Interleukin-6 is a cytokine which plays a role both in inflammatory reactions and in the maturation of B lymphocytes. Furthermore, it has been demonstrated that the substance is an endogenous substance with inflammatory action, a so-called pyrogen, that can trigger a high fever in the event of autoimmune diseases or infections. The protein is predominantly generated at sites of acute or chronic inflammation, from where it is secreted into serum and triggers an inflammatory reaction via the interleukin-6 receptor alpha. Interleukin-6 is involved in various disease states associated with inflammation, including a predisposition for diabetes mellitus or systemic juvenile idiopathic arthritis (Still's disease). The human IL-6 gene is located in chromosome band 7p15.3 and consists of six exons. The IL-6 precursor protein consists of 212 amino acids. After a signal peptide of 28 amino acids in length has been cleaved off, the mature interleukin-6 has a length of 184 amino acids (Hirano T, Yasukawa K, Harada H, Taga T, Watanabe Y, Matsuda T, Kashiwamura S, Nakajima K, Koyama K, Iwamatsu A, et al., 1986. Complementary DNA for a novel human interleukin (BSF-2) that induces B lymphocytes to produce immunoglobulin. Nature 324: 73-76).

It has in turn been found that the inclusion of the relative value of the gene expression level of the gene PPAR-gamma and/or IL-6 in the use according to the invention leads to once again improved prognostic or diagnostic results.

Preference according to the invention is given to a use according to the invention, wherein additionally one or more factors selected from the group consisting of BMI, sex, De Ritis ratio, age, FIB4 score, HbA1c value and serum concentration of one or more enzymes, in turn selected from the group consisting of alanine aminotransferase, aspartate aminotransferase, glutamate dehydrogenase, gamma-glutamyl transferase and alkaline phosphatase, are concomitantly related.

In principle, it is also possible to measure the relevant enzyme concentrations in (whole) blood as well.

BMI is the body mass index defined as: body mass [kg]/body height [m]².

The De Ritis ratio in the context of the present application is defined. The ratio of the serum concentrations in [U/I] for the enzymes aspartate aminotransferase and alanine aminotransferase, i.e., AST/ALT (or GOT/GPT) [De Ritis F, Coltorti M, Giusti G. An enzymic test for the diagnosis of viral hepatitis; the transaminase serum activities. Clin Chim Acta. 1957; 2(1): 70-4.]

The FIB4 score in the context of this application is defined as follows: FIB-4=(age [years]×AST [U/I])/(thrombocyte count [10⁹/I]×√ALT [U/I]) [Sterling R K, Lissen E, Clumeck N, Sola R, Correa M C, Montaner J, S Sulkowski M, Torriani F J, Dieterich D T, Thomas D L, Messinger D, Nelson M; APRICOT Clinical Investigators. Development of a simple noninvasive index to predict significant fibrosis in patients with HIV/HCV coinfection. Hepatology. 2006; 43(6): 1317-25.]

Serum concentration of aspartate aminotransferase in the context of this text is the concentration of the enzyme aspartate aminotransferase (ASAT; also known as aspartate transaminase or AST; also known as glutamate oxaloacetate transaminase or GOT) determined in serum in the unit U/I.

Serum concentration of alanine aminotransferase in the context of this text is the concentration of the enzyme alanine aminotransferase (ALAT; also known as alanine transaminase or ALT; also known as glutamate pyruvate transaminase or GPT) determined in serum in the unit U/I.

Serum concentration of glutamate dehydrogenase in the context of this text is the concentration of the enzyme glutamate dehydrogenase (GLDH or GDH) determined in serum in the unit U/I.

Serum concentration of gamma-glutamyl transferase in the context of this text is the concentration of the enzyme gamma-glutamyl transferase (γ-GT) determined in serum, heparin plasma or EDTA plasma in the unit U/I.

Serum concentration of alkaline phosphatase in the context of this text is the concentration of alkaline phosphatases (AP) determined in serum in the unit U/I.

HbA1c value in the context of this application is the percentage of glycated hemoglobin in total hemoglobin.

It has been found that relating the relative gene expression level of the gene of HMGA2 to one or more of the factors mentioned here leads to a further improved prognostic or diagnostic statement. It is of course preferred in the context of the present text that furthermore one or more of the values of the relative gene expression level of the genes mentioned above are also included.

Preference according to the invention is given to a use, wherein the relative value of the gene expression level or the relative values of the gene expression levels are determined in vitro from an adipose tissue sample.

In the preferred use according to the invention, indirect identification of the composition of the adipose tissue of the individual composed of mature and immature adipose cells and of the functionality thereof and, possibly, degree of inflammation is achieved on the basis of gene expression analysis. In this case, for example—without being tied to these theories-low levels of PPAR-gamma expression and high levels of HMGA2 expression indicate an increased proportion of immature adipose cells in adipose tissue. Conversely, high PPAR-gamma expression and low HMGA2 expression indicate an increased proportion of mature adipose cells in adipose tissue. Low expression of ADIPOQ indicates relatively low functionality of adipose tissue. This may be due to an overwhelming proportion of immature adipose cells, or the adipose cells are in a hypertrophic state, i.e., the adipose cells are overloaded with triglycerides and are no longer capable of producing adiponectin protein encoded by ADIPOQ. High IL-6 expression is associated with a high degree of inflammation processes in adipose tissue, whereas low IL-6 expression levels are more likely to indicate anti-inflammatory processes in adipose tissue. By combining the gene expression of these genes, it is possible to make statements about adipose tissue composition, functionality and inflammatory processes in the adipose tissue of the individual. This combination of values can be used to draw surprisingly accurate conclusions about, inter alia, liver damage, liver fibrosis progression and the presence of NASH.

Preference is given to a use according to the invention, wherein the relative value of the gene expression level or the relative values of the gene expression level are determined in vitro from an adipose sample, wherein the sample was obtained by puncture of abdominal adipose tissue, preferably subcutaneous abdominal adipose tissue.

“In vitro” means that, in the context of the present text, the actual sample collection from the subject is not part of the use according to the invention or the method according to the invention (see below).

By means of fan-shaped punctures under suction, it is possible to obtain preferably cells and cell clusters which allow molecular genetics analysis. Firstly, the fan-shaped puncture procedure reduces clogging/blockage of the cannula tip with adipose cells and, secondly, what are obtained are cells from various regions of the adipose tissue in question and thus a representative cross-section of the distribution of different cell types of adipose tissue.

Particular preference is given to a use according to the invention, wherein the sample mass for the samples from adipose tissue is ≤100 mg, preferably ≤50 mg, more preferably ≤20 mg and even more preferably ≤5 mg.

It has been found that, surprisingly, differentiated results can be reliably achieved even with very small sample volumes from adipose tissue. In this connection, it is particularly preferred that the sample was obtained by puncture as fine-needle aspirate.

The determination of the various parameters from adipose tissue is considered by the inventors to also have the following prognostic advantages: Firstly, according to the invention, it is possible, after the determination of the parameters from adipose tissue, to identify individuals already suffering from NAFLD or NASH (see also below). Secondly, the inventive determination of the parameters from adipose tissue allows earlier identification of individuals who have an increased probability of developing NAFLD, NASH or liver fibrosis compared to, for example, conventional FIB4 score values or the De Ritis ratio (see also below).

Preferably according to the invention, the subject is a human, since a differentiated prognosis and diagnosis in the case of liver diseases in humans is of very particular importance both in relation to the economy and in relation to health policy.

As already indicated above, it is preferred that the determination of the gene expression level is done at the mRNA level. Thus, it is possible to obtain reliable data using extremely low sample amounts and by means of established methods.

Preference is given to a use according to the invention, wherein the value or values for the relative gene expression levels and optionally one or more factors, as mentioned above, are related using the multivariate model of self-organizing maps by Kohonen (SOM).

The following describes statistical methods which, alone or in combination, are suitable for relating in the context of the present invention. This includes a description of the SOM method:

The goal of the applied method is to create classifications (clusters) of liver diseases on the basis of various biomarkers such as HMGA2, ADIPOQ, IL-6 or PPAR-gamma and of markers described above that extend the prior classifications for diagnostic, but also future therapeutic purposes.

To formally describe the study data, the customary methods of descriptive statistics are used. For nominal parameters, absolute frequency and relative frequency are specified, and for ordinal parameters, the median is additionally specified. For metric values, mean value and standard deviation are calculated. Normal distributions are tested with the aid of the Kolmogorov-Smirnov test (KS test). Nonparametric correlations between the biomarkers are calculated with the aid of Kendall's tau-b. For comparisons between categorical variables, the χ2 test is used.

To calculate the a priori unknown clusters, self-organizing maps (SOM) are used. SOMs (in this case, Kohonen maps by Teuvo Kohonen, cf. Teuvo Kohonen: Self-Organizing Maps. Springer-Verlag, Berlin 1995, ISBN 3-540-58600-8) are types of artificial neural networks having an unsupervised learning method with the goal of achieving a topographic feature map in the form of clusters of the input space (patient data). Here, patients (subjects) within a cluster are intended to be maximally homogeneous and, between the clusters, maximally inhomogeneous. SOMs are used for clustering, visualizing complex relationships, prediction (evaluation), modeling and data exploration. The network used here consists of 1000 neurons, correlations are automatically compensated and missing values are taken into account. To produce the clusters, the SOM-WARD clustering method is used (2-stage hierarchical cluster algorithm). Color codings are carried out using heat maps. The clusters produced as a result are compared descriptively. To describe the clusters with the aid of decision trees or facts and rules, various classification algorithms are used, such as C5.0, CART and Exhausted Chaid. As a measure of quality for the various classifiers, what were assessed were classification accuracy, compactness of the model (e.g., size of a decision tree), interpretability of the model, efficiency and robustness in the face of noise and missing values.

To further validate the models and to calculate the importance of the biomarkers for the various classification models, RBF networks (radial basis function networks) are created as a prediction model. The RBF networks yield a suitable approximation of the cluster assignment of the SOMs. The input vectors are normalized (subtraction of the mean value and division by the range (x-Min)/(Max-Min); normalized values are in the range between 0 and 1). The activation function used is the softmax function σ as normalized radial basis function. Softmax σ maps a k-dimensional vector z onto a k-dimensional vector σ(z).

The network performance (how “good” the network is) is checked on the basis of the following data:
Model summary: Results including error, relative error or percentage of false predictions.
Classification results: A classification table was specified for each dependent variable.
ROC curves: ROC curves (Receiver Operating Characteristic curves) specify the sensitivity and specificity for each possible cut-point of the input variables. The Area under the Curve AUC is a measure of the quality of the classification, and also
Cumulative gain charts.

Specifically, the following methods are used:
1. Self-organizing neural networks (→Kohonen maps)
2. Classification algorithms
   Entropy-based learning methods (C5.0)
   Exhausted Chaid and
   CART
3. Radial basis functions (specific type of neural networks)
4. Descriptive and inductive statistics

### 1) Self-Organizing Neural Networks

Self-organizing maps (SOM) refer to types of artificial neural networks having an unsupervised learning method with the goal of achieving a topological representation of the input space (in this case, patient data). The best-known SOMs are the topology-maintaining Kohonen maps by Teuvo Kohonen. The learning algorithm independently produces classifiers, according to which it divides the input patterns into (hitherto unknown) clusters. The goal is to achieve maximum homogeneity of the patients within a cluster and maximum inhomogeneity of the patients between the clusters.

Core concept (topographic feature map): “Neighboring” input vectors (in this case, patient data) should belong to neighboring neurons in the map, such that the density and distribution of the neurons correspond to the probability model of the training set.

Advantages: Neighborhood relationships in the “confusing” input space can be directly read in the output layer.

Uses: SOMs are used for clustering, visualizing complex relationships, prediction (evaluation), modeling and data exploration. Clustering, visualization and prediction are the focus of what is used for the problems present here.

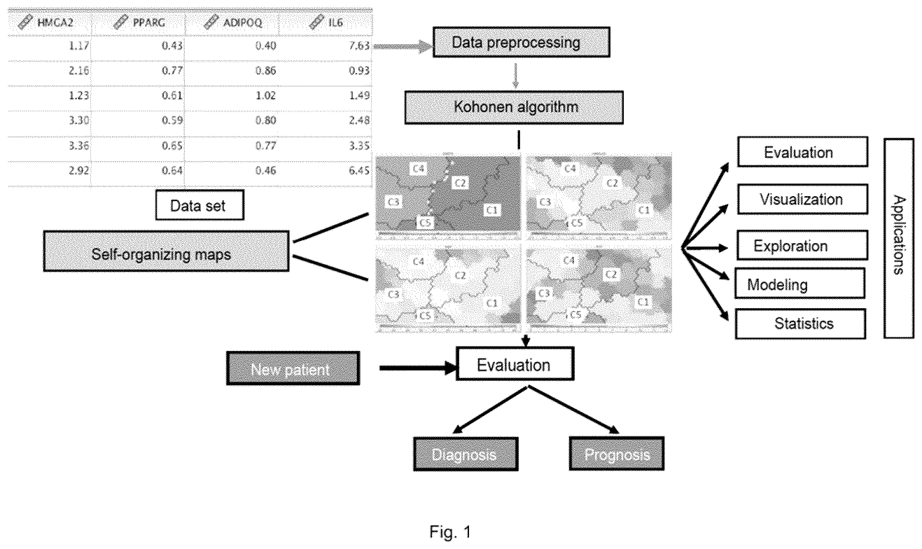

(Tools: for example, Self Organizing Maps in R (R is a free programming language for statistical calculations and graphs. R is part of the GNU project, cf. also https://cran.r.project.org/web/packages/som/som.pdf and FIG. 1)

### 1.1) Formal Description of the Kohonen Network Model (Algorithm)

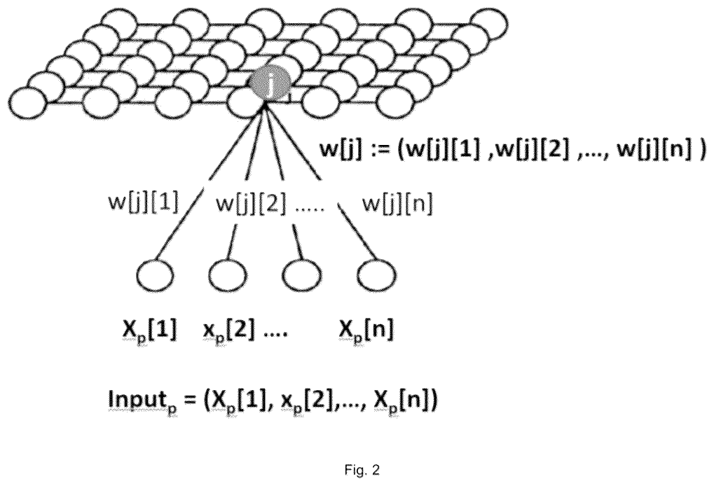

An SOM consists of two layers of neurons (input layer and output layer), cf. also FIG. 2.
Each neuron of the input layer is connected to each neuron of the output layer (the neurons of the input layer are fully networked with those of the output layer). Each neuron of the input layer corresponds to a parameter of the data set. The number of input neurons is the dimension of the input layer. The output neurons are related to one another by a neighborhood weighting function.
The strength of the connection is represented by a number (=weight) w[i][j] (w[i][j] is the weight which specifies the strength of the connection between the i-th input neuron and the j-th output neuron). The vector w[j] represents all weights w[i][j] (i=1 . . . n, n is the number of parameters acquired for each patient) in relation to the j-th output neuron. Input vectors and weight vectors are normalized (length=1).
Initialize the weight vectors. Arbitrary values produced by a random generator are specified for the weights as start default.
Each patient p defines, by the values thereof, an input vector Inputₚ=(xₚ[1], xₚ[2], . . . , Xₚ[n]) with the components Inputₚ[i]=xₚ[i]. These input vectors are first normalized to 1.
For each patient p and each neuron j in the output layer, the Euclidean distance

$${s}_{p} [ j ] = ‖ w [ j ] - {input}_{p} ‖ = \sqrt{{\sum }_{i} {( w [ j ] [ i ] - {input}_{p} [ i ] )}^{2}}$$

between the weight vector w[j] and the input vector Inputₚ is calculated.
The output neuron which has the smallest distance to the input vector Inputₚ is called the winner neuron (“winner takes all”). The weight vector of the winner neuron is most similar to the input vector, i.e., it has the “maximum stimulation” under the input vector Inputₚ. If 2 or more output neurons should have the same minimum distance, one neuron is selected by a random generator.
The winner neuron and its neighbors are awarded the “contract”, i.e., may represent the input.
A function f(inputₚ) is hereby defined which assigns a location a in a representative layer (map) to each vector inputₚ of the input space (pattern space, feature space)
f: inputₚ→f(inputₚ).=arg min(∥w[j]−inputₚ∥). The minimum is formed over all weight vectors w[j]. The function arg provides the index of the winner neuron.
Determine for each patient (input pattern) the winner neuron according to the above instructions.
Neighborhood weighting function and weight adaption: In the next step, the weights of the winner neuron and those of its surrounding neurons are adapted. In this case, the degree of neighborliness in relation to the winner neuron plays a large role. Let us assume the winner neuron has the index. For an input[i] and an output neuron j, Dw[j][i] refers to the weight change in the context of a learning rule. This is calculated as follows:

$$Dw [ j ] [ i ] = η ( t ) * ( input [ i ] - w [ i ] [ j ] ) * NbdWt ( α , j )$$

where
η(t) refers to the learning rate (η(t) where 0<η(t)<1) and
NbdWt(α, j) refers to the neighborhood weighting function. A neighborhood weighting function calculates the degree of neighborliness of an output neuron in relation to the winner neuron, which is between 0 and 1. Convergence evidence against a statistically describable equilibrium state exists for, for example, η(t):=η*t^{-a} where 1<a≤1.
Approximate weight vectors to one another proportionally to the neighborhood weighting function The weight vectors w[j] of all neurons j are updated according to the neighborhood weighting function NbdWt(α, j) of the neuron α. The new weight w_{new}[i][j] is calculated as follows:

$${w}_{new} [ j ] [ i ] : = w [ j ] [ i ] + Dw [ j ] [ i ]$$

The neighborhood weighting function can, for example, be one of the following functions:

$$NbdWt ( α , j ) = \frac{1}{1 + {( \frac{d ( α , j )}{s} )}^{2}}$$

$$NbdWt ( α , j ) = {e}^{- {( \frac{d ( α , j )}{s} )}^{2}}$$

where d(α, j):=(α−j); s is a scalar. The width s=s(t) of the neighborhood weighting function and the learning step width η(t) are reduced over time: The winner neuron and its neighbors are “activated” according to the function NbdWt(α, j).
Normalize weight vectors to a length of 1, present next data point.
For the various methods for map color codings (heat maps), reference is made to the literature (e.g.,
http://citeseerx.ist.psu.edu/viewdoc/download?doi=10.1.1.100.500&rep=rep1&type=pdf,
https://arxiv.org/pdf/1306.3860.pdf
or
https://www.visualcinnamon.com/2013/07/self-organizing-maps-creating-hexagonal.html).

### 2) Classification Algorithms

To evaluate the SOM models (description of the classes by facts and rules or decision trees), 3 different classification algorithms are used: (1) Entropy-based learning methods (C5.0), (2) Exhausted Chaid and (3) CART.

### 2.1) Definition.

Classification methods are methods and criteria for classifying objects (in this case, patients) into classes (in this case, types and subtypes of healthy prediabetics and diabetics).

From a training set of examples having known class affiliation, a classifier in the form of decision trees or equivalent in the form of facts and “If-Then” rules is generated with the aid of the classification algorithm. Classification is differentiated from clustering (see also SOMs) in that the classes are a priori known in classification, whereas the classes must first be sought in clustering.

Decision trees serve for decision making by means of an arboreal structure consisting of a root node (start node), nodes, edges and leaves (end nodes) (FIG. 2).

Formally, a tree is a finite graph having the properties:
1) There is exactly one node in which no edge ends (the “root”).
2) Exactly one edge ends in each node different from the root, the edges are directed
3) Each node is reachable from the root on exactly one path.

A decision tree is a tree:
1) Each node tests an attribute
2) Each branch corresponds to an attribute value
3) Each leaf (node without outgoing edges) assigns a class.

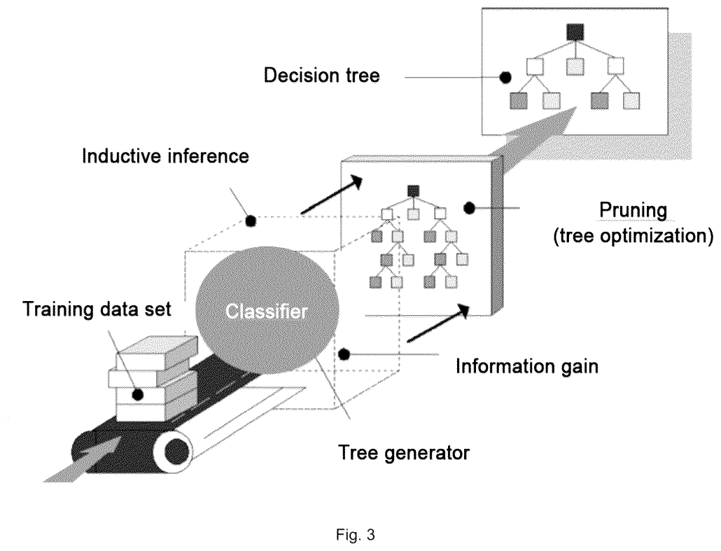

FIG. 3 shows the formal components of a classification algorithm.

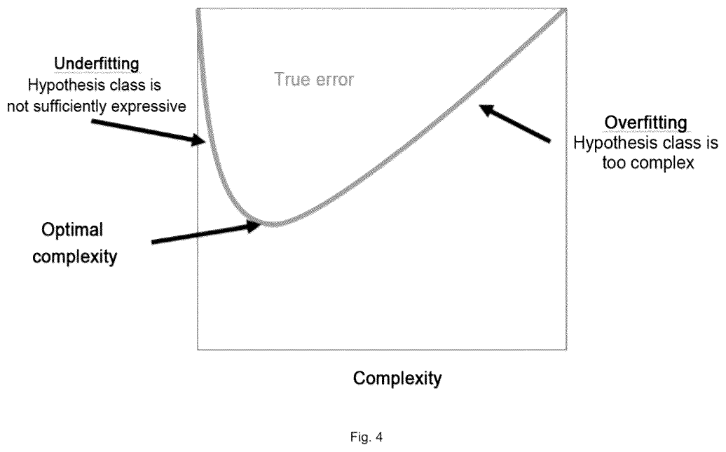

Problem: The classifier is optimized for the training data in the first step. It may possibly provide relatively poor results (underfitting or overfitting: hypothesis class is too inexpressive or too complex) on the data population, cf. FIG. 4.

A possible overfitting can be reduced by pruning or boosting methods; in this case, a person skilled in the art chooses the number of required iteration steps to improve group formation.

In general, the set of available examples is divided into two subsets (train-and-test).
Training set: for the learning of the classifier (construction of the model)
Test set: for the assessment of the classifier

If this is not usable because the set of objects having known class affiliation is small, the so-called m-fold cross-validation is used instead of train-and-test.

The following criteria are taken as measures of quality for classifiers:
Classification accuracy.
Compactness of the model (e.g., size of a decision tree)
Interpretability of the model.
Efficiency.
Robustness in the face of noise and missing values

### 2.2) Construction of Decision Trees

(See also
Quinlan, J. Ross (1986): Induction of decision trees. In: Machine Learning 1 (1), pages 81-106.
Quinlan, J. Ross (1993): C4.5. Programs for machine learning. In: J. Ross Quinlan. San Mateo, Calif.: Morgan Kaufmann (The Morgan Kaufmann series in machine learning).
Quinlan, J. Ross (1996): Improved use of continuous attributes in C4. 5. Journal of artificial intelligence research 4, pages 77-90.)

### Basis Algorithm

### Loop:

1. Choose the “best” decision attribute A for the next node
2. For each value of A, generate a new child node
3. Assign the training data to the child nodes
4. If the training data are classified without errors, then STOP. Otherwise, iterate over child nodes (→1.).

### Designations:

Training data set T,
Number of training data |T|,
Classes Cᵢ: The data set of all training data in the class Cᵢ. |Cᵢ| is the number of elements in class Cᵢ. The following is valid: Σ|Cᵢ|=|T| (i=1, . . . k).
Attribute A={α₁, α₂, . . . , αₘ}. Attribute A subdivides the data set T into m subsets T₁, T₂, . . . , Tₘ. |Tᵢ| is the number of subset Tᵢ.
Given training data set T and attributes A;
Output: Information gain (T, A) of attributes A for training data set T
   3) Algorithm (Calculation of Information Gain with the Aid of Entropy Using the Example of ID3)
The empirical entropy for a set T of training objects having the classes Cᵢ (i=1, . . . , k) is defined as

$$entropy ( T ) = {\sum }_{i = 1}^{k} {p}_{i} \cdot log {p}_{i}$$

$$where {p}_{i} := \vert {C}_{i} \vert / \vert T \vert$$

The attribute A has produced the partitioning T₁, T₂, . . . , Tₘ. The empirically determined entropy G(T,A) for a set T and an attribute A is defined as

$$G ( T , A ) = {\sum }_{i = 1}^{m} \frac{\vert {T}_{i} \vert}{\vert T \vert} \cdot entropy ( {T}_{i} )$$

The information gain Gain(T, A) due to the attribute A in relation to T is defined as

$$Gain ( T , A ) = entropy ( T ) - G ( T , A ) = entropy ( T ) - {\sum }_{i = 1}^{m} \frac{\vert {T}_{i} \vert}{\vert T \vert} \cdot entropy ( {T}_{i} )$$

Input: Training data set T having the classes Cᵢ (i=1, . . . , k), attributes A and threshold ε;
Output: Decision Tree E;

### Algorithm:

1. Creation of a node K;
2. If all example data in T have an identical class Cⱼ or the number of data is smaller than threshold ε, the single node is back as a leaf with class Cⱼ in relation to node K;
3. If A=Ø, the single node is assigned as a leaf with the most common class in T in relation to node K;
4. Calculation of the information gain of A in T and determination of the best attribute A_{g} with maximum information gain on the basis of

$$Gain ( T , A ) = entropy ( T ) - G ( T , A ) = entropy ( T ) - {\sum }_{i = 1}^{m} \frac{\vert {T}_{i} \vert}{\vert T \vert} \cdot entropy ( {T}_{i} )$$

5. Designation of node K with A_{g}
6. For all attribute values A_{gi} of A_{g}, calculate the sub-data set T_{gi} of all examples from the training data set with A_{gi}
7. If T_{gi}=Ø, a leaf with the most common class in relation to node K is added, otherwise
8. Recursion of the branch of A_{g}.

### Note:

For a random event y which occurs with probability P(y), the following applies:

h(y)≡−log₂(P(y)) Information content:

The entropy is the average value of the information content of the random variable y

$$H [ y ] ≡ \sum y P ( y ) h ( y ) = - \sum y P ( y ) log 2 ( P ( y ) )$$

### Classification Algorithms Used

### ID3

Discrete attributes, no missing attributes
Information gain as measure of quality.
Extension of ID3.
Information gain ratio as measure of quality. Missing attributes.
Numerical and real-valued attributes.
Pruning of the decision tree

The information gain ratio is defined as

$$Gain Ratio ( T , A ) := \frac{Gain ( T , A )}{SplitInfonnation ( T , A )}$$

$$SplitInfonnation ( T , A ) = - {\sum }_{i = 1}^{m} \frac{\vert {T}_{i} \vert}{\vert T \vert} \cdot {log}_{2} ( \frac{\vert {T}_{i} \vert}{\vert T \vert} )$$

### CART (Classification and Regression Trees)

See also Hastie, T., Tibshirani, R., Friedman, J. H. (2001). The elements of statistical learning: Data mining, inference, and prediction. New York: Springer Verlag.

Method analogous to ID3 or C5.0. The measure of information is defined by the Gini index. The Gini index is minimized (instead of maximizing the Gini gain).

### Gini Gain

$$Gini ( T , A ) = {\sum }_{i = 1}^{k} \frac{{T}_{i}}{T} \cdot Gini ( {T}_{i} )$$

$$where Gini ( T ) = 1 - {\sum }_{i = 1}^{k} {p}_{i} 2$$

Pᵢ is the relative frequency of the class Cᵢ in T

### CHAID (Chi-Square Automatic Interaction Detectors)

See also Sonquist, J. A. and Morgan, J. N. (1964): The Detection of Interaction Effects. Survey Research Center, Institute for Social Research, University of Michigan, Ann Arbor.

CHAID is another algorithm for constructing decision trees. The differences in relation to C5.0 or CART are that the chi-square test of independence is used to choose the attributes in the CHAID algorithm and that the CHAID algorithm stops the growth of the tree before the tree has become too large. The tree is thus not left to grow at will in order to shorten it again afterwards with a pruning method.

### 4) Radial Basis Function Networks (RBF Networks)

See also Zell, A.: “Simulation neuronaler Netze” [simulation of neural networks]. Oldenbourg 1994

To further validate the models and to calculate the importance of the biomarkers for the various classification models, RBF networks are used. RBF networks create prediction models. They are particularly suitable for the approximation of functions.

The RBF network consists of an input layer having n neurons, a hidden layer having k neurons and an output layer having m neurons. An n-dimensional pattern is mapped thereby into an m-dimensional output space.

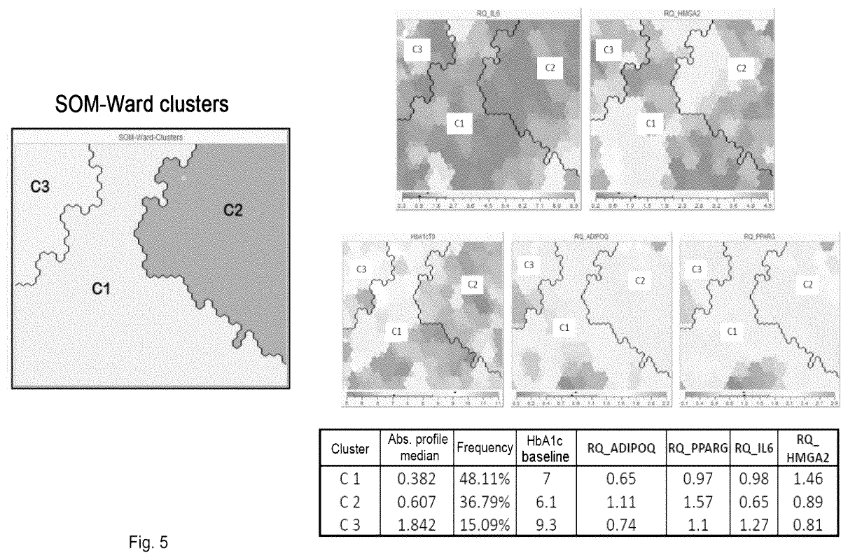

The input layer is purely a forwarding means. Each neuron distributes its value to all hidden neurons. In the hidden layer, in each neuron, the distance between the input and the center c is formed with the aid of a Eucledean norm. Radial basis functions are used as network input and activation function (cf. FIG. 5).

The activation function of each hidden neuron is a so-called radial function,
i.e., a monotonically decreasing function

$$f : {IR}_{0}^{+} \to [ 0 , 1 ] where f ( 0 ) = 1 and {lim}_{x \to ∞} f ( x ) = 0 m$$

The input vectors are normalized (subtraction of the mean value and division by the range (x-Min)/(Max-Min); normalized values are in the range between 0 and 1). The activation function used is the softmax function σ as normalized radial basis function. Softmax o maps a k-dimensional vector z onto a k-dimensional vector σ(z).

$$σ {( z )}_{j} = \frac{{e}^{{𝓏}_{j}}}{{\sum }_{k = 1}^{K} {e}^{{𝓏}_{k}}} , j = 1 , … , k$$

The number of neurons in the hidden layer is determined by the “Bayesian Information Criterion” (cf. Schwarz, Gideon E. (1978), “Estimating the dimension of a model”, Annals of Statistics, 6 (2): 461-464, MR 468014, doi:10.1214/aos/1176344136) (BIC). The best number of hidden units is that which yields the smallest BIC on the basis of the training data.

For the output layer, we used the identity function as activation function. Thus, the output units are singly weighted sums of the hidden units. The output of the network is therefore a linear combination of the radial basis functions of the inputs and the weights.

### Network Performance

Network performance checks how “good” the network is. To this end, a series of results is provided.
Model summary.
Results including error, relative error or percentage of false predictions and training time.
Classification results.
A classification table is specified for each categorical dependent variable.
ROC curves.
The ROC curves (Receiver Operating Characteristic curves) specify the sensitivity and specificity for each possible cut-point of the input variables. The Area under the Curve AUC is a measure of the quality of the classification.
Cumulative gain charts.

Preference is given to a use according to the invention, wherein the use is carried out to accompany or to prepare for a bariatric procedure.

A bariatric procedure in the context of this text is any surgical procedure used as an anti-obesity measure (bariatric surgery). These include in particular proximal Roux-en-Y gastric bypass and sleeve gastrectomy, but also biliopancreatic diversion with or without duodenal switch, gastric band implantation, omega loop gastric bypass and other procedures.

It has been found that, surprisingly, the prognostic or diagnostic results of the use according to the invention are very precise in connection with a bariatric procedure.

Preference is given to a use according to the invention, which involves distinguishing between NAFLD and NASH especially in the case of diagnosis.

According to the current prior art, secure distinguishing between nonalcoholic fatty liver disease (NAFLD) and nonalcoholic steatohepatitis (NASH) is only possible with particular reliability by means of liver biopsy (see also above).

It has now been found that, surprisingly, the use according to the invention is capable of appropriately distinguishing the particular subject with a high degree of certainty on the basis of samples, in particular adipose samples.

Preference according to the invention is given to a use according to the invention, wherein a prognosis of deterioration of the state of the liver, in particular the onset of new liver fibrosis or the progression of preexisting liver fibrosis, is made.

It has been found that an appropriate prognosis is reliably possible with the use according to the invention.

This is especially the case in combination with a bariatric procedure.

Preference is given to a use according to the invention, wherein the prognosis of deterioration of the state of the liver is related to the probability of type 2 diabetes remission, in particular after a bariatric operation.

This preferred use according to the invention not only allows the prognosis of deterioration of the state of the liver, but also—as has been shown surprisingly—makes it possible to achieve a prediction of type 2 diabetes remission with good results. This too is especially the case in combination with bariatric procedures on the patient.

Preference is given to a use for prognosis according to the invention, wherein the sample investigated is assigned to one of the following three risk clusters:
C1: Increased probability of type 2 diabetes remission and reduced risk of liver fibrosis;
C2: Increased probability of type 2 diabetes remission and increased risk of liver fibrosis;
C3: Reduced probability of type 2 diabetes remission and reduced risk of liver fibrosis

“Risk groups” in the context of this text are those groups which can be separated from one another through suitable distinguishing features and have in each case a common increased or nonincreased risk with regard to the development or the presence of a disease, in particular NAFLD, NASH, liver fibrosis, liver cirrhosis and hepatocellular carcinoma. Moreover, risk groups can be additionally distinguished from one another by further physiological differences, and this may have therapeutic or prophylactic relevance.

It is surprising that the use according to the invention accordingly allows complex predictions not only in relation to the prognosis of liver fibrosis, but also in relation to the prognosis of type 2 diabetes remission.

In general, the determination of the relative expression values (see above) requires use of a calibrator value for the respective gene or for gene expression. In case of doubt, a person skilled in the art can choose the following calibrator values for delta Ct for the following genes:

TABLE 1

| Gene | Delta Ct, rounded |
|---|---|
| ADIPOQ | 7.770 |
| HMGA2 | 7.321 |
| IL-6 | 0.858 |
| PPARG | 11.150 |

Regarding the markers listed in the table below, the respective clusters are provided with the mean value entered in the table, though of course the fluctuation in a patient population means that the respective values are to be seen as values with +−10%. However, the distribution pattern of the values in the three clusters (highest mean HMGA2 expression in cluster 1, highest mean ADIPOQ and PPARG expression in cluster 2, highest mean HbA1c value in cluster 3) is independent thereof.

TABLE 2

| Marker | C1 | C2 | C3 |
|---|---|---|---|
| RQ ADIPOQ | 0.65 | 1.11 | 0.74 |
| RQ HMGA2 | 1.46 | 0.89 | 0.81 |
| RQ IL-6 | 0.98 | 0.65 | 1.27 |
| RQ PPARG | 0.97 | 1.57 | 1.10 |
| HbA1c T0 [%] | 7.0 | 6.1 | 9.3 |

The mean values in bold in Table 2 are particularly striking for describing the clusters.

In relation to this, reference is also made in principle to the examples.

Preference is given to a use according to the invention, wherein the liver disease is selected from the group consisting of NAFLD, NASH, liver fibrosis, liver cirrhosis and hepatocellular carcinoma.

The use according to the invention has been found to be particularly suitable for these disease forms.

The invention also includes a method for prognosing and/or diagnosing a liver disease, comprising the steps of:
a) providing in vitro a sample from a subject,
b) determining the relative value of the gene expression level of the gene HMGA2 and/or of a gene, the expression of which linearly statistically correlates with that of the gene HMGA2, and
c) relating the value determined in step b) to one or more gene expression level values as further defined above and/or to one or more factors as further defined above, and
d) taking into account the result of step c), classifying the subject into a group with a disease profile, in the case of diagnosis, or into a group with a risk profile, in the case of prognosis.

In this connection, the steps can also be used in preferred methods according to the invention (in case of doubt likewise preferably) as described above for the use according to the invention.

In the method according to the invention, it is possible to prognose or diagnose liver diseases.

Preferably, it is possible to additionally make a statement about type 2 diabetes remission (see above); this is especially the case in combination with a bariatric procedure.

The advantages described above in relation to the use according to the invention of course also apply to the methods according to the invention in a corresponding embodiment.

The invention will be further elucidated below using examples:

### EXAMPLE 1

### Prediction of Risk of Advanced Liver Fibrosis by Means of Gene Expression Analyses of HMGA2, PPAR-Gamma, ADIPOQ and IL-6 in Subcutaneous Adipose Tissue Samples

### Materials and Methods

### Tissue Samples

The human subcutaneous abdominal adipose tissues were collected during operations and stored in liquid nitrogen after the operation. For all human adipose tissue samples used, the requirements of the Declaration of Helsinki were met. A written declaration of consent for the use of the tissue samples was returned by the patients (n=106).

### RNA Isolation

Total RNA was isolated by means of an RNeasy Lipid Tissue Mini Kit (QIAGEN, Hilden, Germany) in a QIAcube (QIAGEN, Hilden, Germany) according to the manufacturer's instructions. The tissue samples (up to 100 mg) in 1 ml of QIAzol Lysis Reagent were homogenized in a gentleMACS Octo Dissociator (Miltenyi Biotec, Bergisch Gladbach, Germany), centrifuged and transferred to a fresh 2 ml reaction tube, and the homogenate was then incubated at room temperature for 5 min. This was followed by the addition of 200 μl of chloroform, which was mixed with the sample by vigorous shaking by hand for 15 sec. The sample was incubated again at room temperature for 2 min and centrifuged at 12 000×g at 4° C. for 15 min. Thereafter, the upper aqueous phase was transferred to a fresh 2 ml cup and the total RNA was isolated over a Qiagen RNeasy Mini Spin column (QIAGEN, Hilden, Germany) in a QIAcube according to the manufacturer's instructions.

cDNA Synthesis

For the cDNA synthesis, ≤250 ng of RNA was transcribed into cDNA by means of 200 U of M-MLV reverse transcriptase, RNase Out (Invitrogen, Darmstadt, Germany) and 150 ng of random primers (Invitrogen, Darmstadt, Germany) according to the manufacturer's instructions. The RNA was denatured at 65° C. for 5 min and subsequently stored on ice for at least 1 min. After the addition of the enzyme, the mix was incubated at 25° C. for 10 min for the annealing of the random primers to the RNA. The subsequent reverse transcription was carried out at 37° C. for 50 min, followed by a 15 min inactivation of the reverse transcriptase at 70° C.

cDNA Preamplification

5 μl of cDNA was preamplified by means of PerfeCTa PreAmp SuperMix (Quantabio, Beverly, Massachusetts, USA) using HMGA2- and HPRT- (hypoxanthine phosphoribosyltransferase 1) specific primers according to the manufacturer's instructions. The cDNA was preamplified according to the following temperature profile: 95° C. for 2 min followed by 14 cycles at 95° C. for 10 sec and at 60° C. for 3 min.

Quantitative Real-Time PCR (qRT-PCR)

The relative quantification of gene expression was carried out by means of real-time PCR on the Applied Biosystems 7300 Real-Time PCR System. Commercially available gene expression assays (Life Technologies, Carlsbad, CA, USA) were used for the quantification of the mRNA level of HMGA2 (assay ID Hs00171569_m1) and PPAR-gamma (assay ID Hs01115513_m1). As described by Klemke et al. (Klemke M, Meyer A, Hashemi Nezhad M, Belge G, Bartnitzke S, Bullerdiek J (2010) Loss of let-7 binding sites resulting from truncations of 3′ untranslated region of HMGA2 mRNA in uterine leiomyomas. Cancer Genet Cytogenet 196:119-123), HPRT was used as endogenous control. All measured samples were determined in triplicate. Gene expression was quantified in 96-well plates containing the preamplified cDNA to be investigated, the respective gene-specific assay and the FastStart Universal Probe Master (Rox) (Roche, Mannheim, Germany). The temperature profile of the real-time PCR followed the manufacturer's instructions: The template is denatured at 95° C. for 10 min. This was subsequently followed by amplification in 50 cycles, starting with denaturation at 95° C. for 15 sec and the combination of annealing/elongation at 60° C. for 60 sec. The data obtained were evaluated by means of a comparative delta Ct method (ΔΔCT method). [(Livak K J, Schmittgen TD (2001) Analysis of relative gene expression data using real-time quantitative PCR and the 2^{-ΔΔC(T)} Method. Methods 25:402-408)]

### Results

There is increasing clinical and mechanistic evidence of the positive effects on metabolism and weight loss due to bariatric surgery with regard to improving nonalcoholic fatty liver disease (NAFLD) in obese patients. Bariatric surgery is associated with a significant improvement in both histological and biochemical markers of NAFLD. Studies in respect of bariatric surgery and liver diseases show a significant reduction in the incidence of a number of histological features of NAFLD, including steatosis, fibrosis, hepatocytic ballooning and lobular inflammation. A bariatric operation is also associated with a reduction in liver enzyme values, with a statistically significant reduction in ALT, AST and gamma-GT values [Bower G, et al.: Bariatric Surgery and Non-Alcoholic Fatty Liver Disease: a Systematic Review of Liver Biochemistry and Histology; Obes Surg. 2015 December; 25(12):2280-9. doi: 10.1007/s11695-015-1691-x]. In contrast, studies however also show that excessively rapid weight loss due to a bariatric procedure can lead to distinct deterioration of existing steatohepatitis [Andersen T, Gluud C, Franzmann M B, Christoffersen P. Hepatic effects of dietary weight loss in morbidly obese subjects. J Hepatol 1991; 12:224-9.; Luyckx F H, Scheen A J, Desaive C, Dewe W, Gielen J E, Lefebvre P J. Effects of gastroplasty on body weight and related biological abnormalities in morbid obesity. Diabetes Metab 1998; 24:355-61.]. Currently, there is no method that can identify individuals who will experience deterioration of their liver disease following a bariatric procedure.

A study by the inventors examined a patient cohort who underwent a bariatric operation for the influence of the procedure after one year on remission of their type 2 diabetes (T2D) and their liver values. For this purpose, self-organizing maps (SOMs) were used to carry out statistical evaluation of the parameters gene expression of HMGA2, PPAR-gamma, ADIPOQ and IL-6 in subcutaneous abdominal adipose tissue and HbA1c value at the time of the operation. In this study, it was found that these parameters and statistical analysis can be used to identify patient subgroups which experience no remission (C3) and experience T2D remission because of this bariatric procedure. Surprisingly, two different subgroups (C1 and C2), each of which experiences T2D remission, were identified. However, with regard to an improvement in liver values and in NAFLD after one year, in particular liver fibrosis, it was found that particularly patients in the T2D remission cluster C2 are more likely to experience deterioration of their liver fibrosis than the other two clusters C1 and C3.

The FIB4 score can be used to estimate the risk of advanced liver fibrosis. The FIB4 score is calculated according to the following formula

$$FIB 4 score = ( age \times AST ) / ( thrombocyte count \times ( √ ( ALT ) ) .$$

In the case of low values (<1.3 in patients under 65 years of age, <2.0 in patients over 65 years of age), there is no increased risk of advanced fibrosis, but in the case of values >3.25, the risk is increased. Intermediate values require further investigations in order to be able to estimate the risk.

The FIB4 score was calculated for 106 patients and then classification was carried out into the three categories “low risk for advanced fibrosis”, “advanced fibrosis unclear” and “high risk for advanced fibrosis”. Bariatric procedures were performed on all 106 patients. Twelve months after these procedures, the FIB4 scores were calculated again. It was found here that eight patients were assigned to a category with a more favorable risk prediction than at the time of the procedure. 93 out of 106 patients initially fell into the low-risk category. One year after the surgical procedure, it was necessary on the basis of the FIB4 score to assign 45 patients (corresponding to 48.4%) with an initially low risk to a category with a less favorable risk prediction with respect to advanced liver fibrosis.

TABLE 3
Cross-table initial FIB4 categories versus FIB4 categories after 12 months

| | | FIB4 category after 12 months | | | |
|---|---|---|---|---|---|
| | | Low risk for | Advanced | High risk for | |
| | | advanced | fibrosis | advanced | |
| | | fibrosis | possible | fibrosis | Total |
| Initial FIB4 | Low risk for | 48 | 39 | 6 | 93 |
| category | advanced fibrosis | 51.6% | 41.9% | 6.5% | 100.0% |
| | Advanced fibrosis | 7 | 5 | 0 | 12 |
| | possible | 58.3% | 41.7% | 0.0% | 100.0% |
| | High risk for | 0 | 1 | 0 | 1 |
| | advanced fibrosis | 0.0% | 100.0% | 0.0% | 100.0% |

| Total | 55 | 45 | 6 | 106 |
|---|---|---|---|---|
| | 51.9% | 42.5% | 5.7% | 100.0% |

The possible existence of a link between the parameters initial BMI (at the time of surgery) and expression of the genes HMGA2, PPARG, ADIPOQ and IL-6 (each determined at the time of surgery) and the deterioration of the FIB4 score after 12 months was investigated.

The FIB4 score observed after 12 months does not appear to be dependent on the initial BMI or the expression of the genes PPARG, ADIPOQ and IL-6, or appears to have comparatively little dependence thereon. In contrast, it was found that deterioration of the FIB4 score after 12 months occurs predominantly in patients who initially had relatively low HMGA2 expression.

Besides the FIB4 score, it therefore appears appropriate to determine HMGA2 expression in subcutaneous adipose tissue, especially when performing bariatric procedures. With the aid thereof, it is possible as early as at the time of surgery to identify those patients whose FIB4 score will deteriorate within one year and who are thus exposed to an increased risk of progressive liver fibrosis. If such risk patients are identified on the basis of reduced HMGA2 expression, they can be monitored more closely, for example by means of elastography, in order to be able to detect at an early stage the possibly severe manifestation of liver fibrosis.

To this end, a cluster analysis of the 106 patients was carried out using Kohonen self-organizing maps (SOMs). The parameters considered were the initial HbA1c value at the time of the surgical procedure and the expression levels of the genes HMGA2, PPARG, ADIPOQ and IL-6. This approach yielded three clusters of patients characterized by different characteristics.

FIG. 5 a shows the summarized result in the SOM analysis. There are three distinct clusters C1 to C3. FIGS. 5b to 5f show how the parameters expression of IL-6 and HMGA2, initial HbA1c value and expression of the genes ADIPOQ and PPARG are manifested in the three clusters. In relation to this, the table specifies the mean values determined in the respective clusters.

Cluster C1 is distinguished by increased HMGA2 expression, whereas the genes PPARG and ADIPOQ are more strongly expressed in cluster C2. Cluster C3 is characterized by high initial HbA1c values.

To assign the patients to the three clusters, the following eight rules are calculated using the C5.0 algorithm and are required for the classification of the patients:

TABLE 4
Rules for assigning the patients to one of three clusters.

| No. | Rule | Cluster assignment |
|---|---|---|
| 1 | RQ HMGA2 > 2.0 | C1 |
| 2 | RQ ADIPOQ <= 0.9 | C1 |
| 3 | HbA1c <= 7.2 and RQ HMGA2 <= | C2 |
| | 2.0 and RQ PPARG > 1.2 | |
| 4 | HbA1c <= 7.2 | C2 |
| 5 | HbA1c > 7.2 and RQ HMGA2 <= 1.6 | C3 |
| | and RQ ADIPOQ > 0.9 | |
| 6 | HbA1c > 7.2 and RQ PPARG <= 0.6 | C3 |
| 7 | HbA1c > 9.5 and RQ HMGA2 <= 1.6 | C3 |
| 8 | HbA1c > 7.2 and RQ HMGA2 <= 0.3 | C3 |

The clusters differ with respect to type 2 diabetes remission: whereas patients assigned to cluster C3 do not experience remission following the bariatric procedure, patients of both clusters 1 and 2 experienced remission one year after surgery.

TABLE 5
Classification of patients with T2D remission after one year

| | Cluster C3 | Cluster C1 + C2 | % correct |
|---|---|---|---|
| Actually observed | No | Yes | predictions |
| No | 16 | 1 | 94.1% |
| Yes | 0 | 89 | 100.0% |
| Proportion | 15.1% | 84.9% | 99.1% |

It becomes apparent that a very high accuracy of prediction can also be made with respect to T2D remission by means of the applied methods on the basis of gene expression levels: In the entire patient population of 106 individuals, the prediction derivable from the SOM model was anomalous only in a single case, insofar as T2D remission occurred in group C3 contrary to expectations.

In case of doubt, precisely the positive prognosis of an ensuing T2D remission is an important decisive factor for an operation.

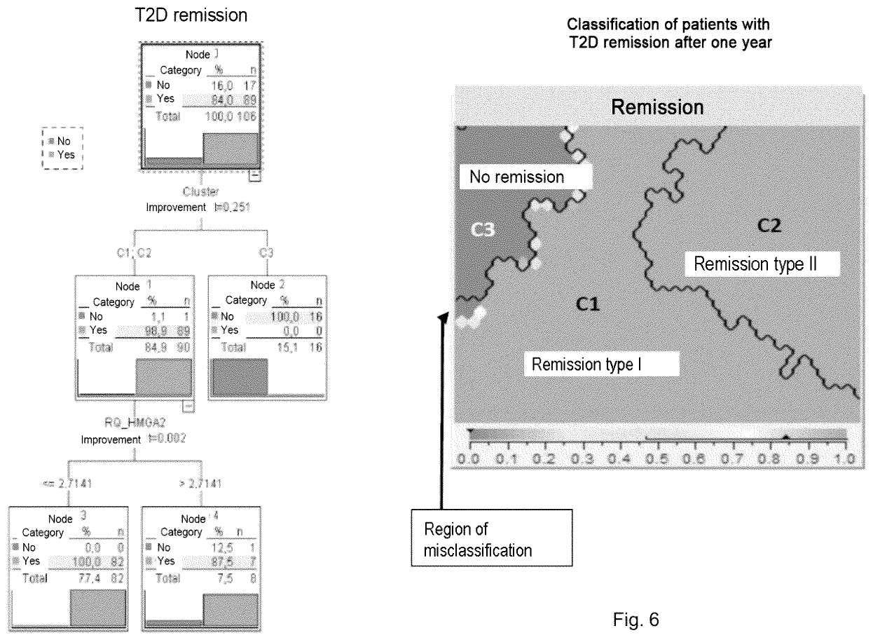

The corresponding SOM pattern is also shown in FIG. 6.

Furthermore, significant differences in the clusters were found with regard to the risk for advanced liver fibrosis one year post-surgery. This risk is lower in both cluster C1 (with remission) and cluster C3 (no remission), i.e., the majority of patients in these clusters (62.7%) are at low risk. In contrast, the second cluster with remission, C2, is associated with more patients with an increased risk for advanced liver fibrosis (66.7%).

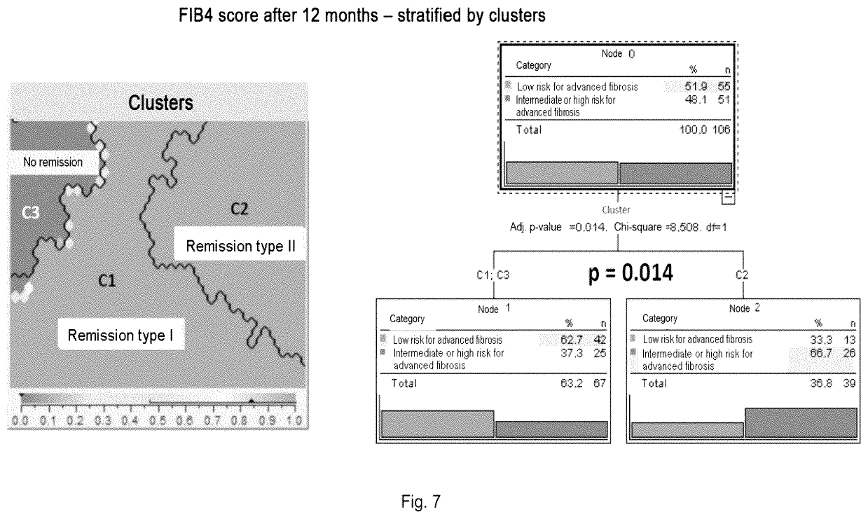

FIG. 7 shows the result of the SOM evaluation with respect to FIB4 development after twelve months.

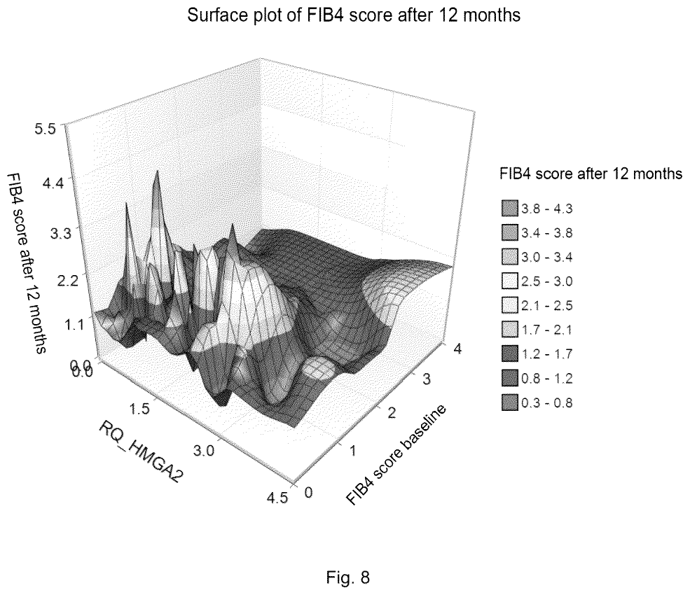

In addition, FIG. 8 shows a surface plot of the FIB4 score after twelve months as a function of the relative expression level of HMGA2 and initial FIB4 score. This shows the significant influence of HMGA2 on the clusters achieved.

### EXAMPLE 2

### Estimation and Prognosis of Severe Liver Damage on the Basis of the Expression Levels of the Genes HMGA2, PPARG, ADIPOQ and IL-6

### Materials and Methods

### Sample Preparation

The tissue samples were obtained as described in Example 1. The following steps of sample preparation, i.e., RNA isolation, cDNA synthesis, cDNA preamplification and quantitative real-time PCR, were also carried out as described in Example 1.

### Statistical Analysis

See Example 1 (differences mentioned here)

### Results

The De Ritis ratio is used to estimate the severity of liver damage and is calculated as follows: De Ritis ratio=AST/ALT. Values <1 indicate less pronounced liver damage, whereas values >1 indicate more severe liver damage (e.g., chronic hepatitis, liver cirrhosis). The De Ritis ratio determined one year after a bariatric procedure is significantly dependent on the value established at the time of surgery.

However, statistical analysis showed that the De Ritis ratio to be established one year after a bariatric procedure is affected by not only the initial De Ritis ratio, but also the initial BMI, age and sex and also the expression levels of the genes HMGA2, PPARG, ADIPOQ and IL-6.

The following features were included in the statistical analysis carried out:

### Biomarkers:

RQ HMGA2 (77.4%),
RQ ADIPOQ (49.1%),
RQ IL-6 (11.3%),
RQ PPAR-gamma (2.8%)
and as basic characteristics
sex (46.4%)
initial De Ritis ratio (70.8%)
age (60.34%)
initial BMI (50.0%)

The values between parenthesis each indicate the weighted influence of the individual marker in the evaluation result.

As a consequence, the evaluation resulted in 78 patients being classified into the class with relatively low liver damage after twelve months (cluster A), whereas 28 patients were classified into cluster B with liver damage expected to be relatively high (after twelve months in each case).

The evaluation after twelve months revealed misclassification of only two patients from cluster A and only four patients from class B. Thus, the applied system makes it possible to make a comparatively good prediction of development within a period of twelve months.

It is also noteworthy here again that the relative expression level of HMGA2 had the greatest influence on group assignment.

Table 6 below shows the decision tree for classification.

TABLE 6
Prediction of severeness of liver damage after 12 months:

| | | Severeness of |
|---|---|---|
| No. | Rule | liver damage |
| 1 | De Ritis ratio, initial < 1 | Low |
| | RQ HMGA2 <= 4.1 | |
| | RQ ADIPOQ <= 1.3 | |
| | BMI, initial > 35 | |
| | BMI, initial <= 65 | |
| | Age > 35 | |
| | Sex = female | |
| 2 | De Ritis ratio, initial < 1 | Low |
| | RQ HMGA2 <= 0.8 | |
| | BMI, initial <= 65 | |
| | Sex = female | |
| 3 | De Ritis ratio, initial < 1 | Low |
| | RQ HMGA2 > 0.8 | |
| | Sex = male | |
| 4 | RQ ADIPOQ <= 1.5 | Low |
| | Age <= 40 | |
| | Sex = male | |
| 5 | Age > 60 | Low |
| 6 | RQ PPARG <= 1.2 | Severe |
| | BMI, initial > 65 | |
| | Sex = female | |
| 7 | De Ritis ratio, initial >= 1 | Severe |
| | RQ IL6 > 0.4 | |
| | Age <= 60 | |
| 8 | RQ HMGA2 > 4.1 | Severe |
| 9 | De Ritis ratio, initial < 1 | Severe |
| | RQ HMGA2 > 0.8 | |
| | RQ ADIPOQ > 1.3 | |
| 10 | BMI, initial <= 35 | Severe |
| 11 | RQ HMGA2 <= 0.8 | Severe |
| | Sex = male | |

### EXAMPLE 3

Distinguishing between NAFLD and NASH on the basis of the expression levels of the genes HMGA2, PPARG, ADIPOQ and IL-6 in subcutaneous adipose tissue

### Materials and Methods

### Sample Preparation

The tissue samples were obtained as described in Example 1. The following steps of sample preparation, i.e., RNA isolation, cDNA synthesis, cDNA preamplification and quantitative real-time PCR, were also carried out as described in Example 1.

### Statistical Analysis

See Example 1 (differences indicated here)

### Results

Liver biopsy is still the gold standard for diagnosing NAFLD, since NASH can only be formally diagnosed by histological means. However, biopsy is invasive and carries the risk of potentially life-threatening complications such as bleeds.

Investigations were also carried out on a data set of 52 cases to determine whether the stages of nonalcoholic steatohepatitis (NASH) and nonalcoholic fatty liver disease (NAFLD) can be distinguished.

For this purpose, the parameters cluster assignment, FIB4 score and expression levels of the genes HMGA2, PPARG, ADIPOQ and IL-6 were used and a rule set for classification of the patients was developed using the C5.0 algorithm. The following eight rules are required to assign the patients to the NASH group or NAFLD group:

TABLE 7
Rules for distinguishing between
NASH patients and NAFLD patients.

| No. | Rule | Assignment |
|---|---|---|
| 1 | Initial FIB4 score <= 0.3 | NAFLD |
| 2 | RQ HMGA2 <= 0.3 | NAFLD |
| 3 | RQ PPARG <= 0.9 and RQ IL-6 <= 0.3 | NAFLD |
| 4 | Cluster C2 and RQ ADIPOQ > 1.0 | NAFLD |
| 5 | Initial FIB4 score > 0.3 and cluster C1 and | NASH |
| | RQ HMGA2 > 0.3 and RQ PPARG > 0.9 | |
| 6 | Cluster C3 | NASH |
| 7 | Initial FIB4 score > 0.35 and cluster C1 and | NASH |
| | RQ HMGA2 > 0.27 and RQ IL-6 > 0.27 | |
| 8 | Initial FIB4 score > 0.35 and cluster C2 and | NASH |
| | RQ ADIPOQ <= 0.96 | |

These rules correctly determined the stage of liver disease in 49 out of 52 cases, which corresponds to a 5.8% error rate. One case of NAFLD was incorrectly identified as NASH, and two cases of NASH were incorrectly assigned to the NAFLD group.

The NAS score (NAFLD activity score) is a hitherto important tool for assessing fatty liver diseases. Since it is based on histological criteria, a liver biopsy must be carried out, which is associated with certain risks (e.g., bleeds). This invasive procedure can be replaced with the system described here, in which a sample is harmlessly collected from the subcutaneous adipose tissue.

We claim:

1. The use of the relative value of the gene expression level of the gene HMGA2 and/or of a gene, the expression of which statistically correlates with that of the gene HMGA2, in the prognosis and/or diagnosis of a fatty liver disease of a subject.

2. The use as claimed in claim 1, wherein the relative value of the gene expression level of the gene HMGA2 and/or of a gene, the expression of which statistically correlates with that of the gene HMGA2, is related to the relative value of the gene expression level of the gene ADIPOQ and/or of a gene, the expression of which statistically correlates with that of the gene ADIPOQ.

3. The use as claimed in claim 1, wherein additionally the relative value (i) of the gene expression level of the gene HMGA2 or (ii) of the gene HMGA2 and of the gene ADIPOQ is related to the relative value of one gene expression level or preferably both gene expression levels of the genes selected from the group consisting of PPAR-gamma and IL-6.

4. The use as claimed in claim 1, wherein additionally one or more factors selected from the group consisting of BMI, sex, De Ritis ratio, age, FIB4 score, HbA1c value and serum concentration of one or more enzymes, in turn selected from the group consisting of alanine aminotransferase, aspartate aminotransferase, glutamate dehydrogenase, gamma-glutamyl transferase and alkaline phosphatase, are concomitantly related.

5. The use as claimed in claim 1, wherein the relative value of the gene expression level or the relative values of the gene expression levels are determined in vitro from an adipose tissue sample.

6. The use as claimed in claim 5, wherein the sample was obtained by puncture of abdominal adipose tissue, preferably subcutaneous abdominal adipose tissue.

7. The use as claimed in claim 1, wherein the determination of the gene expression level or gene expression levels is done at the mRNA level.

8. The use as claimed in claim 1, wherein the value or values for the relative gene expression levels and optionally one or more factors, as mentioned in claim 4, are related using the multivariate model of self-organizing maps by Kohonen.

9. The use as claimed in claim 1, wherein the use is carried out to accompany or to prepare for a bariatric procedure.

10. The use as claimed in claim 1, which involves distinguishing between NAFLD and NASH.

11. The use as claimed in claim 1, wherein a prognosis of deterioration of the state of the liver, in particular the onset of new liver fibrosis or the progression of pre-existing liver fibrosis, is made.

12. The use as claimed in claim 11, wherein the prognosis of deterioration of the state of the liver is related to the probability of type 2 diabetes remission.

13. The use as claimed in claim 12, wherein the sample investigated is assigned to one of the following three risk clusters:
C1: Increased probability of type 2 diabetes remission and reduced risk of liver fibrosis;
C2: Increased probability of type 2 diabetes remission and increased risk of liver fibrosis;
C3: Reduced probability of type 2 diabetes remission and reduced risk of liver fibrosis.

14. The use as claimed in claim 1, wherein the liver disease is selected from the group consisting of NAFLD, NASH, liver fibrosis, liver cirrhosis and hepatocellular carcinoma.

15. A method for prognosing and/or diagnosing a liver disease, comprising the steps of:
a) providing in vitro a sample from a subject,
b) determining the relative value of the gene expression level of the gene HMGA2 and/or of a gene, the expression of which statistically correlates with that of the gene HMGA2, and
c) relating the value determined in step b) to one or more gene expression level values as further defined in either of claims 2 and 3 and/or to one or more factors as further defined in claim 4, and
d) taking into account the result of step c), classifying the subject into a group with a disease profile, in the case of diagnosis, or into a group with a risk profile, in the case of prognosis.
